(19) United States
(12) Patent Application Publication
(10) Pub. No.: US 2005/0256312 A1
(43) Pub. Date: Nov. 17, 2005

(54) Process for preparing 4-substituted 2,2,6,6-tetramethylpiperidin-N-oxy and 2,2,6,6-tetramethylpiperidin-N-hydroxy compounds

(75) Inventors: Clemens Osterholt, Dorsten (DE); Heinz-Gunter Poll, Marl (DE); Oliver Meyer, Munster (DE); Thomas Kubelback, Dulmen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Degussa AG, Duesseldorf (DE)

(21) Appl. No.: 11/125,149

(22) Filed: May 10, 2005

(30) Foreign Application Priority Data
May 10, 2004 (DE) 102004023640.2

(51) Int. Cl. C07D 211/36; C07D 491/10

(52) U.S. Cl. 546/16; 546/242

(57) ABSTRACT

A process for preparing 4-substituted 2,2,6,6-tetramethylpiperidin-N-oxy compounds (II) or mixtures of (II) and 4-substituted 2,2,6,6-tetramethylpiperidin-N-hydroxy compounds (III) 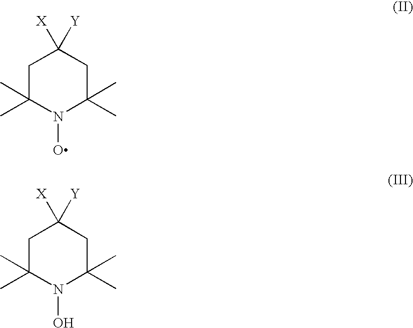
where X+Y can be O or can represent a cyclic ketal with the radicals 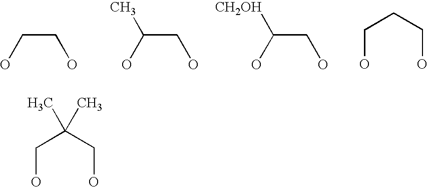
or can represent an open-chain ketal in which X═O—R and Y═O—R′, where R and R′ can be identical or different and can each be CH₃, CH₂—CH₃, CH₂—CH₂—CH₃, CH(CH₃)—CH₃, CH₂—CH₂—CH₂—CH₃ and CH₂—CH(CH₃)—CH₃,
by oxidizing corresponding 4-substituted 2,2,6,6-tetramethylpiperidines (I) 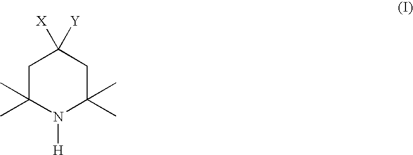 with hydrogen peroxide in the presence of alkali metal hydrogencarbonate and/or ammonium hydrogencarbonate and in the presence or absence of a solvent, in which the reaction is carried out with the addition of Brönsted acids which have an acid strength greater than that of the hydrogencarbonate.

### BACKGROUND OF THE INVENTION

1. Field of the Invention

The invention relates to a process for preparing 4-substituted 2,2,6,6-tetramethyl-piperidin-N-oxy compounds (II) and 4-substituted 2,2,6,6-tetramethylpiperidin-N-hydroxy compounds (III) by oxidizing corresponding 4-substituted 2,2,6,6-tetramethylpiperidines (I) with hydrogen peroxide in the presence of one or more hydrogencarbonate salts as catalyst, according to the following equation (A) 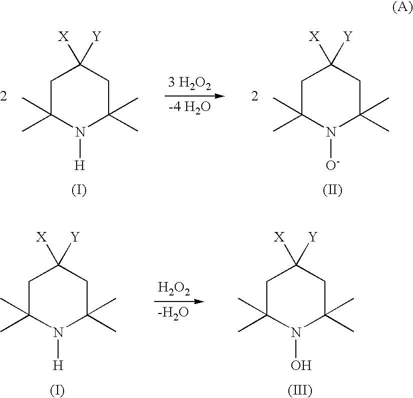
where X+Y can be O or can represent a cyclic ketal with the radicals 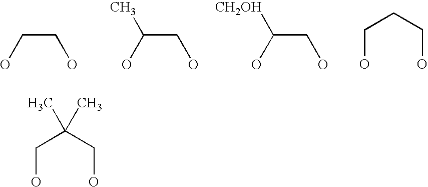
or can represent an open-chain ketal in which X═O—R and Y═O—R′, where R and R′ can be identical or different and can each be CH₃, CH₂—CH₃, CH₂—CH₂—CH₃, CH(CH₃)—CH₃, CH₂ CH₂—CH₂—CH₃ and CH₂—CH(CH₃)—CH₃.

2. Description of the Related Art

Owing to their properties, stable N-oxyl radicals, also known as TEMPO derivatives, are widely used as oxidation catalysts, as polymerization inhibitors or as mass regulators in free-radical polymerizations. In particular, 2,2,6,6-tetramethyl-4-oxopiperidin-N-oxy (also referred to as 4-oxo-TEMPO or TEMPON for short) is used for stabilizing unsaturated monomers. The N-oxyl radical 2,2,6,6-tetramethyl-4-oxopiperidin-N-oxy is of the following formula (II) where X+Y are a single oxygen atom.

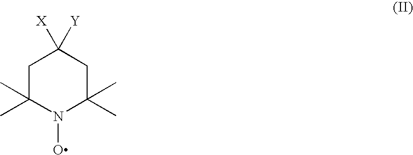

A number of methods for the oxidation of, for example, triacetonamine (TAA) to the corresponding stable N-oxyl radical 4-oxo-TEMPO are known from the literature.

Oxidants used for this reaction are, inter alia, persulfate (Tetrahedron Lett. 1995, 36, 31, 5519-5522), persulfonic acid (Bull.Acad.Sci.USSR Div.Chem.Sci (Engl. Transl.) 1990, 39, 1045-1047), dimethyldioxirane (Tetrahedron Lett. 1988, 29, 37, 4677-4680), ozone (SU963987) and organic hydroperoxides (RU 2139859). The electrochemical oxidation of triacetonamine is also described in the literature (PL148157).

However, in the vast majority of cases, the secondary amine function is oxidized to the N-oxyl group by means of hydrogen peroxide as oxidant (e.g. Tetrahedron 1992, 48, 9939-9950; Chemical Papers 1988, 42, 2, 243-248; Tetrahedron 1985, 41, 1165-1172; J. Prakt. Chem. 1985, 327, 6, 1011-1014; Chem. Pharm. Bull. 1980, 28, 3178-3183; US 5817824). In contrast to the above-mentioned oxidants, hydrogen peroxide has the great advantage that only water and possibly oxygen are formed as coproducts.

The methods which have hitherto been described in the literature for the oxidation of TAA by hydrogen peroxide thus differ mainly in respect to the oxidation catalysts which are used to facilitate oxidation.

The use of sodium tungstate as oxidation catalyst is most frequently described in the literature (Tetrahedron 1992 48, 9939-9950; Chemical Papers 1988, 42, 2, 243-8; Tetrahedron 1985, 41, 1165-1172; J. Prakt. Chem. 1985, 327, 6, 1011-1014, U.S. Pat. No. 5,817,824). Disadvantages are the sometimes unsatisfactory yields and also, inter alia, the relatively high price and the contamination of wastewater with tungsten salts.

Some of these processes make additional use of, for example, the alkali metal salts of ethylenediaminetetraacetic acid (EDTA), for example in U.S. Pat. No. 5,817,824, mainly because of their basic nature and because their complexing properties probably contribute to the stability of hydrogen peroxide toward heavy metal ions (U.S. Pat. No. 5,817,824, column 10, lines 32 to 36).

Nevertheless, yields of only 40.8-58% of theory are achieved.

The Italian patent application IT 2000MI1052 describes a process in which triacetonamine derivatives are reacted with hydrogen peroxide to form the corresponding N-oxy derivatives and in which exclusively phosphonic acids or their salts, e.g. heptasodium diethylenetriaminepentamethylenephosphonate, are used as catalysts. In the conversion of triacetonamine into 4 oxo-TEMPO under discussion here, the conversion is 93.3% but the selectivity is only 67.6%, which corresponds to a yield of only 63% (see Example 7 of IT 2000MI1052). Although the corresponding ketals are mentioned, no examples or yields are given for these. In addition, the water-soluble phosphonic acids represent impurities for particular applications and may also be undesirable in the wastewater and, because of their high price, make the production process expensive.

The use of sodium carbonate or sodium hydrogencarbonate as catalysts has also been described, e.g. in Dokl. Chem. (Engl. Transl.) 1981, 261, 466-467 (corresponds to Dokl. Akad. Nauk SSSR Ser. Kim. 1981, 261, 109-110). Although this method is very simple, it has the disadvantage that very long reaction times of 4-5 days are necessary for a crude yield of 95% and relatively high excesses of 2.7 equivalents of hydrogen peroxide are consumed.

In the repetition of the same process described in J. Prakt. Chem. 1985, 327, 6, 1011-1014, a yield of only 73% of theory was obtained in a corresponding fashion after a reaction time of two days. In addition, the method has been described only for a glass vessel and is not, as has been found here, suitable for use in industrial apparatuses which are usually constructed of metal.

U.S. Pat. No. 5,629,426 describes a process specifically for the preparation of 4-hydroxy-2,2,6,6-tetramethylpiperidine N-oxide by oxidation of the corresponding amine in the presence of carbonate or bicarbonate and EDTANa₂ as chelating agent. This serves first and foremost to scavenge traces of iron or other metals from the reaction mixture during the production process and in this way prevents destruction of hydrogen peroxide. However, EDTA is also undesirable as impurity for particular applications. In addition, owing to its high price, it likewise makes the production process expensive. Furthermore, nothing is said about whether the process is successful in the case of the above-mentioned TAA derivatives or in reactors having metal surfaces. However, on the basis of the description, in particular the batch sizes of <1 mol of substrate, it may be assumed that what is being described is laboratory examples carried out in glass apparatuses which thus do not allow reliable statements to be made about the ability of the process to be implemented in industry.

EP 574 666 describes only an oxidation method for ketals of triacetonamine which is carried out only in glass apparatus and in which divalent metal salts of alkaline earth metals and zinc are used as catalysts, i.e. likewise a method which cannot be carried out directly in industrial metal apparatuses.

On an industrial scale in particular, a high H₂O₂ consumption and poor conversion of the substrate to an oxidized product are caused by a number of factors. In particular, impurities such as heavy metal ions play an important role in the decomposition of H₂O₂. Likewise, the decomposition of H₂O₂ increases with increasing reactor surface area and increasing roughness of the reactor surface, since traces of heavy metals can be continually dissolved from the wall of the vessel at such irregularities. This can be countered to a certain degree by appropriate passivation of the reactor before commencement of the reaction. The stirrer speed also plays a not inconsiderable role and an excessively high temperature can likewise promote decomposition of H₂O₂.

Likewise, a high pH, which can result, for example, from an excessively high proportion of Na₂CO₃, leads to increased H₂O₂ decomposition.

The decomposition of NaHCO₃ used, for example, as catalyst according to the equation 2 NaHCO₃→Na₂CO₃+H₂O+CO₂ can also lead to increased formation of Na₂CO₃, with the disadvantageous consequences mentioned. It has also been found that increased decomposition of hydrogen peroxide leads to increased formation of Na₂CO₃, which once again accelerates the decomposition of hydrogen peroxide as a result of the above-mentioned pH effect.

### SUMMARY OF THE INVENTION

Accordingly, it is an object of the invention to provide a very simple process which can be carried out on an industrial scale for preparing the above-mentioned 4-substituted 2,2,6,6-tetramethylpiperidin-N-oxy compounds (II) or mixtures of (II) with 4-substituted 2,2,6,6-tetramethylpiperidin-N-hydroxy compounds (III) by oxidizing corresponding 4-substituted 2,2,6,6-tetramethylpiperidines (I) by means of hydrogen peroxide, which does not have the above-mentioned disadvantages and which can be carried out without problems and preferably without additional equipment in conventional industrial metal apparatuses.

In particular, the process should be able to be carried out using very low excesses or consumptions of reactants and at the same time very short reaction times while giving high space-time yields on an industrial scale, too, and make possible a very simple form of purification preferably without expensive or toxicologically dangerous auxiliaries or critical waste streams and also be able to give a very high yield and purity of target product.

These and further objects which are not explicitly mentioned but can readily be derived or concluded from the relationships discussed herein are achieved by a process as set forth in the claims. Advantageous embodiments and modifications of the process of the invention are further claimed.

### DETAILED DESCRIPTION OF THE INVENTION

A process for preparing 4-substituted 2,2,6,6-tetramethylpiperidin-N-oxy compounds (II) or mixtures of (II) and 4-substituted 2,2,6,6-tetramethylpiperidin-N-hydroxy compounds (III) 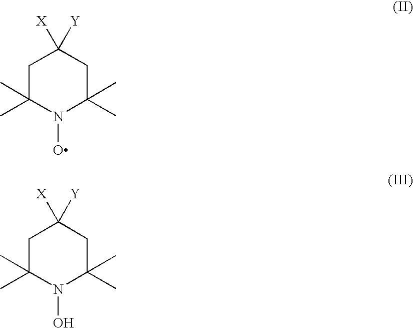
where X+Y can be 0 or can represent a cyclic ketal with the radicals 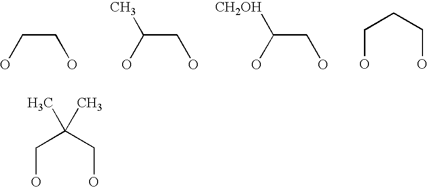
or X+Y can represent an open-chain ketal in which X═O—R and Y═O—R′, where R and R′ can be identical or different and can each be CH₃, CH₂—CH₃, CH₂—CH₂—CH₃, CH(CH₃)—CH₃, CH₂ CH₂—CH₂—CH₃ and CH₂—CH(CH₃)—CH₃,
by oxidizing corresponding 4-substituted 2,2,6,6-tetramethylpiperidines (I) 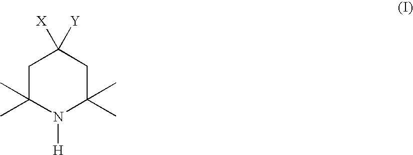
with hydrogen peroxide in the presence of alkali metal hydrogencarbonate and/or ammonium hydrogencarbonate, in the presence or absence of a solvent, and carrying out the reaction with addition of Brönsted acids which have an acid strength greater than that of the hydrogencarbonate of the acid makes it possible, in a very simple fashion, to prepare the 4-substituted 2,2,6,6-tetramethylpiperidin-N-oxy compounds (II) or mixtures of (II) with 4-substituted 2,2,6,6-tetramethylpiperidin-N-hydroxy compounds (III) in high yields and selectivities with at the same time low losses of hydrogen peroxide in industrial apparatuses having metal surfaces without additional equipment on an industrial scale, and to overcome the further disadvantages of the prior art described above.

Where X and Y together represent a single oxygen atom in Formula (II) and (III) the following structures are included

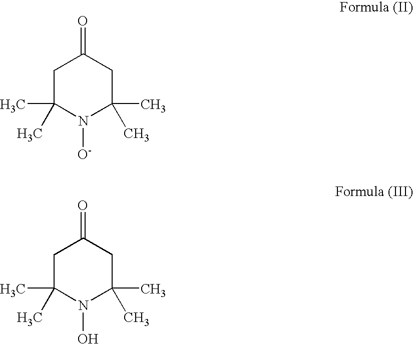

According to the reaction equation (A), it is possible for the invention oxidation of (I) to result in coformation of the analogous N-hydroxy derivatives (III) in varying proportions. However, separation is generally not necessary since the N-hydroxy derivatives can in most cases be used in the same way as the N-oxy derivatives because the two forms can transform into one another in situ. The process of the invention is therefore applicable both to the preparation of the pure N-oxy derivatives (II) and to the preparation of mixtures of (II) and the corresponding N-hydroxy derivatives (III).

However, ≧90 mol % of N-oxy derivatives and, correspondingly, ≦10 mol % of N-hydroxy derivatives are usually formed in the process of the invention, so that either the virtually pure N-oxy derivatives or mixtures comprising at least 90 mol % of N-oxy derivatives are preferably obtained according to the invention.

In the present context, a Brönsted acid is any acid which can release protons. Brönsted acids which have an acid strength greater than that of the hydrogencarbonate of the acid are, according to the invention, those which have a lower pKₐ than hydrogencarbonate. Thus, it is advantageous to use at least one Brönsted acid which has a pKₐ of less than 10.3, preferably ≦9.2, in particular from −1.5 to 7.2 at a temperature of 25° C., in the process of the invention. The determination of the pKₐ of most Brönsted acids are known per se and may be found in standard chemical textbooks.

As alkali metal hydrogencarbonate, preference is given to lithium, sodium, potassium, rubidium and/or cesium hydrogencarbonate, but in particular sodium hydrogencarbonate. Sodium hydrogencarbonate has the particular advantage that it is very inexpensive and readily available.

Hydrogencarbonate serves as catalyst for the reaction and can therefore be used in substoichiometric amounts, preferably in an amount of from 0.02 to 0.5 molar equivalent, but in particular from 0.1 to 0.25 molar equivalent, of alkali metal hydrogencarbonate and/or ammonium hydrogencarbonate, based on the amine (I) to be oxidized.

As Brönsted acid, it is possible to use one or more inorganic or organic acids, or mixtures of inorganic and organic acids.

As inorganic acids, preference is given to phosphoric acid, dihydrogenphosphate and/or hydrogenphosphate, in particular alkali metal or ammonium dihydrogenphosphate, di(alkali metal) or diammonium hydrogenphosphate or alkali metal/ammonium hydrogenphosphate, and also nitric acid, sulfuric acid, alkali metal or ammonium hydrogensulfate and/or hydrohalic acids, in this case particularly preferably hydrochloric acid. All the acids mentioned have, in particular, the advantage that they are all inexpensive, readily available and are mostly unproblematical in wastewater in the amounts which typically occur.

As organic acids, preference is given to using, for example, formic acid or a saturated linear or branched monobasic or polybasic aliphatic carboxylic acid which has from 2 to 12 carbon atoms and may be substituted by O—R¹ or NR²R³, where R¹, R² or R³ can be identical or different and can be hydrogen, an aliphatic or cycloaliphatic, saturated alkyl radical having from 1 to 12 carbon atoms or a carboxyalkyl group (CH₂)ₙCOOH where n=1 to 5 or R² and R³ can together form a saturated, unsubstituted or alkyl-substituted alkylene chain having from 4 to 11 carbon atoms, with the proviso that when R² and R³ are both hydrogen or alkyl or the two together form an alkylene group or when R²=hydrogen and R³=alkyl, the NR²R³-substituted carboxylic acid has to be polybasic.

However, very particular preference is given to using acetic acid, hydroxyacetic acid, methoxyacetic acid, propionic acid, butyric acid, 2-ethylhexanoic acid, oxalic acid, malonic acid, succinic acid, glutaric acid, citric acid, iminodiacetic acid, nitrilotriacetic acid or an acidic amino acid. These have, in particular, the advantage that they are readily available, are mostly inexpensive and readily biodegradable.

In the present context, an acidic amino acid is any amino acid which has at least one more carboxyl group than amino functions in the molecule, in particular aspartic acid, glutamic acid, etc.

Further organic acids which can be used according to the invention, either individually or as a mixture, are phosphonic acids and/or their partial salts having the following general formula:
Z(PO₃HₙM₂₋ₙ)ₘ
where Z represents one or more, linear or branched alkylic radicals or diradicals which have a total of from 1 to 10 carbon atoms and can contain a total of up to 3 nitrogen atoms, m can be from 1 to 6 and n can be 1 or 2 and M is an alkali metal or NH₄.

The formula Z(PO₃HₙM₂₋ₙ)ₘ thus defines a group of phosphonic acids and their partial alkali metal or NH₄ salts, with the partial alkali metal salts preferably being sodium or potassium salts. The partial salts used can be any in which at least one protic hydrogen which does not belong to an ammonium group or to any dialkyleneammonium or trialkyleneammonium group is present in the molecule, thus ensuring that an acid having a sufficient acid strength for the process of the invention is present.

If phosphonic acids and/or their partial salts are used, preference is given to using ethylenediphosphonic acid and its partial sodium, potassium and ammonium salts, with the proviso that at least one protic hydrogen which does not belong to any ammonium group present is present in the molecule; 1-hydroxyethylidene-1,1-diphosphonic acid and its partial sodium, potassium and ammonium salts (HEDP), with the proviso that at least one protic hydrogen which does not belong to any ammonium group is present in the molecule; aminotrimethylenephosphonic acid and its partial sodium, potassium and ammonium salts, with the proviso that at least one protic hydrogen which does not belong to an ammonium group or to a trimethyleneammonium group is present in the molecule; ethylenediaminetetramethylenephosphonic acid and its partial sodium, potassium and ammonium salts (EDTMP), with the proviso that at least one protic hydrogen which does not belong to an ammonium group or to a trimethyleneammonium group is present in the molecule; diethylenetriaminepentamethylenephosphonic acid and its partial sodium, potassium and ammonium salts (DTPMP), with the proviso that at least one protic hydrogen which does not belong to an ammonium group or to a trialkyleneammonium group is present in the molecule; hexamethylenediaminetetramethylenephosphonic acid and its partial sodium, potassium and ammonium salts (HMDTMP), with the proviso that at least one protic hydrogen which does not belong to an ammonium group or to a trialkyleneammonium group is present in the molecule, either individually or as a mixture.

The above-mentioned phosphonic acids have, in addition to their acidic action according to the invention, a strongly chelating action toward heavy metal ions, but are relatively expensive.

For this reason, acids which are not phosphonic acids of the type Z(PO₃HₙM₂₋ₙ)ₘ are usually employed.

It is also possible to employ chelating organic acids such as ethylenediaminetetraacetic acid or diethylenetriaminepentaacetic acid and their respective partial sodium, potassium and ammonium salts or else iminodiacetic acid or nitrilotriacetic acid and their respective partial sodium, potassium and ammonium salts or corresponding mixtures as acids, with the proviso that at least one protic hydrogen which does not belong to an ammonium group or to a dialkyleneammonium or trialkyleneammonium group is present in the molecule. However, the above-mentioned chelating organic acids are likewise relatively expensive.

For this reason, none of the above-mentioned chelating organic acids are usually employed for the purposes of the invention.

According to the invention, it is advantageous to use a total of 0.01-0.5 molar equivalent, but in particular 0.03-0.1 molar equivalent, of the Brönsted acids, based on the amine (I) to be oxidized.

The reaction is preferably carried out so that, after the reaction is complete, the aqueous phase in the case of a two-phase mixture or the aqueous/organic phase in the case of a homogeneous reaction medium (when using appropriate solvents) has a pH of from 7.0 to 10.0. However, the reaction is particularly preferably carried out so that the pH after the reaction is complete is from 8.0 to 9.6, in particular from 8.2 to 9.2, especially from 8.4 to 9.0.

The pH is determined at room temperature using a pH electrode. The pH can be influenced mainly by appropriate choice of the Brönsted acids to be used and their amounts and concentrations, but also by appropriate choice of the hydrogencarbonate used and its amount or concentration. The pH can naturally also be determined at any time during the reaction and subsequently be adjusted by addition of suitable amounts of acid so that the preferred pH range is achieved at the end of the reaction.

According to the invention, preference is given to using hydrogen peroxide as an aqueous solution having a concentration in the range from 10 to 90% by weight, in particular in the range from 20 to 60% by weight, but very particularly preferably from 30 to 50% by weight.

The process of the invention can be carried out either without addition of solvents in aqueous solution or suspension or, under particular circumstances, with addition of solvents such as alcohols, diols, ether compounds, ketones, aliphatic, cycloaliphatic, aromatic and/or araliphatic hydrocarbons which are sometimes advantageous for producing a homogeneous aqueous/organic phase.

As solvent, very particular preference is given to using at least one solvent selected from the group consisting of methanol, ethanol, n-propanol and isopropanol, tert-butanol, isobutanol and n-butanol, methoxyethanol, ethoxyethanol, ethylene glycol, propylene glycol, ethylene diglycol, propylene diglycol, alkyl glycol ethers, 1,3- and 1,4-dioxane, tetrahydrofuran, acetone, heptane, cyclohexane, ethylcyclohexane, toluene and xylene.

The appropriate amount of solvent can easily be determined by a person skilled in the art, e.g. by means of simple tests.

The temperature at which the reaction is carried out can be varied within wide limits. However, the reaction is preferably carried out at from 20° C. to 150° C., in particular from 50° C. to 90° C., but very particularly preferably from 60° C. to 80° C.

In the process of the invention, it is generally advantageous for the amine (I), the alkali metal hydrogencarbonate and/or ammonium hydrogencarbonate, the Brönsted acid and any solvent to be initially charged and then the hydrogen peroxide added to the reaction mixture. This can be done either stepwise or continuously, preferably over a period of from 0.1 to 72 hours, in particular over a period of from 2 to 40 hours, but very particularly preferably from 4 to 10 hours.

However, it is equally possible to introduce all of the abovementioned components simultaneously in the desired ratio in a reactor system continuously. The process of the invention can thus be carried out batchwise or particularly advantageously semicontinuously or continuously.

In the continuous or semicontinuous embodiment, all reactor systems known to those skilled in the art for this purpose can be used, in particular tube reactors and reactor cascades having at least two reactors which can have a stirrer or agitator or combinations thereof.

A particular advantage of the process of the invention is that the reaction can be carried out under conditions under which the reaction medium can come into contact with metal surfaces either continually or only part of the time without large losses of hydrogen peroxide or reduced conversion or selectivity. This is also of particular importance from the point of view of process safety, since uncontrolled decomposition of H₂O₂ can have fatal consequences, especially in an industrial process.

The reaction according to the invention is therefore particularly preferably carried out in the presence of metal surfaces comprising alloys of iron, titanium, zirconium, vanadium, niobium, tantalum, chromium, molybdenum, tungsten, manganese, cobalt, nickel, copper, zinc and/or aluminum as are customarily used for the construction of chemical reactors, so that no apparatuses constructed of special materials and no particular pretreatment of the apparatuses are necessary, which represents a very major advantage of the process.

In addition, the process of the invention has an extraordinary robustness and insensitivity to adverse influences. In addition to a reduced H₂O₂ consumption, the process assures a significant overall improvement in safety.

In addition, it has now surprisingly been found that when dihydrogenphosphate is used, the use of hydrogencarbonate as catalyst can be dispensed with entirely and the sole addition of alkali metal dihydrogenphosphate and/or ammonium dihydrogenphosphate as catalyst still gives comparatively good yields of 4-substituted 2,2,6,6-tetramethylpiperidin-N-oxy compounds (II) or mixtures of (II) and 4-substituted 2,2,6,6-tetramethylpiperidin-N-hydroxy compounds (III). This can be particularly advantageous because the catalytically active hydrogencarbonate can be left out completely.

The reaction can also, if desired, be carried out in the presence of at least one of the above-mentioned solvents.

The present invention also provides a process for preparing 4-substituted 2,2,6,6 tetramethylpiperidin-N-oxy compounds (II) or mixtures of (II) and 4-substituted 2,2,6,6-tetramethylpiperidin-N-hydroxy compounds (III) 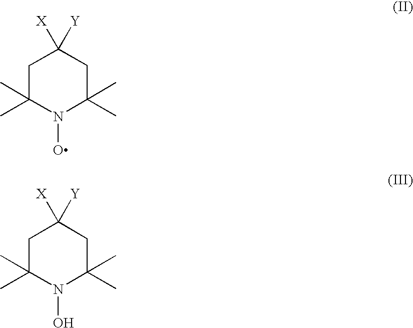
where X+Y can be 0 or can represent a cyclic ketal with the radicals 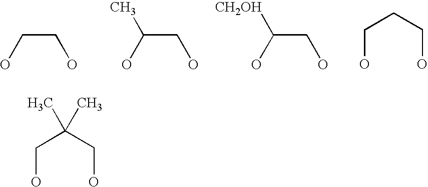
or can represent an open-chain ketal in which X═O—R and Y═O—R′, where R and R′ can be identical or different and can each be CH₃, CH₂—CH₃, CH₂—CH₂—CH₃, CH(CH₃)—CH₃, CH₂ CH₂—CH₂—CH₃ and CH₂—CH(CH₃)—CH₃,
by oxidizing corresponding 4-substituted 2,2,6,6-tetramethylpiperidines (I) 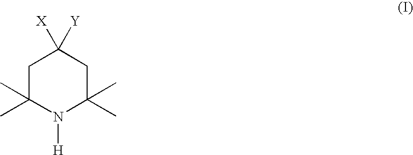
by means of hydrogen peroxide in the absence of hydrogencarbonate, in which the reaction, in the presence or absence of a solvent, is carried out with addition of alkali metal dihydrogenphosphate and/or ammonium dihydrogenphosphate as catalyst.

The reaction is preferably carried out so that the aqueous phase in the case of a two-phase mixture or the aqueous/organic phase in the case of a homogeneous reaction medium has a pH of from 7.5 to 9.0 after the reaction is complete.

The following examples serve to illustrate the invention without further limiting the claims.

### Example 1 (Comparative)

9.5 mol of deionized water, 0.21 mol of NaHCO₃ and 2.0 mol of triacetonamine (TAA) were placed in a jacketed 11 glass reactor provided with a glass blade stirrer, the reaction mixture was brought to a temperature of 60° C. by means of a thermostat while stirring (400 rpm) and 3.5 mol of hydrogen peroxide as a 50% strength aqueous solution were added at a uniform rate via a cool dropping funnel over a period of 4 hours. The reaction temperature was maintained at 57-63° C. The offgas formed in the process, which comprised predominantly O₂, was measured volumetrically by the displacement principle.

At the end of the addition, a sample of the two-phase reaction mixture was taken and the organic phase was analyzed by gas chromatography to determine the product composition. The pH of the lower aqueous phase was measured at room temperature.

### Example 2 (Comparative)

To simulate H₂O₂-destroying conditions, a piece of steel mesh packing was attached to the glass blade stirrer and the experiment was otherwise carried out as in Example 1.

### Example 3

Example 3 was carried out in a manner analogous to Example 2, but 0.06 mol of H₃PO₄ was used as cocatalyst in addition to the 0.21 mol of NaHCO₃ used.

### Example 4

Example 4 was carried out in a manner analogous to Example 2, but 0.06 mol of H₃PO₄ was used as cocatalyst in addition to the 0.21 mol of NaHCO₃ used.

### Example 5

Example 5 was carried out in a manner analogous to Example 2, but 0.06 mol of NaH₂PO₄*1H₂O was used as cocatalyst in addition to the 0.21 mol of NaHCO₃ used.

### Example 6

Example 6 was carried out in a manner analogous to Example 2, but 0.15 mol of Na₂HPO₄*2 H₂O was used as cocatalyst in addition to the 0.21 mol of NaHCO₃ used.

### Example 7

Example 7 was carried out in a manner analogous to Example 2, but 0.05 mol of Na₂HPO₄.2H₂O was used as cocatalyst in addition to the 0.21 mol of NaHCO₃ used.

### Example 8

Example 8 was carried out in a manner analogous to Example 2, but only NaH₂PO₄*1H₂O (0.21 mol) and no NaHCO₃ was used as catalyst.

### Example 9

Example 9 was carried out in a manner analogous to Example 2, but 0.06 mol of nitric acid was used as cocatalyst in addition to the 0.21 mol of NaHCO₃ used.

### Example 10

Example 10 was carried out in a manner analogous to Example 2, but 0.06 mol of H₂SO₄ was used as cocatalyst in addition to the 0.21 mol of NaHCO₃ used.

### Example 11

Example 11 was carried out in a manner analogous to Example 2, but 0.06 mol of hydrochloric acid was used as cocatalyst in addition to the 0.21 mol of NaHCO₃ used.

### Example 12

Example 12 was carried out in a manner analogous to Example 2, but 0.06 mol of acetic acid was used as cocatalyst in addition to the 0.21 mol of NaHCO₃ used.

### Example 13

Example 13 was carried out in a manner analogous to Example 2, but 0.06 mol of 2-ethylhexanoic acid was used as cocatalyst in addition to the 0.21 mol of NaHCO₃ used.

The results of Examples 1-13 are shown in the following table.

| | | | | | Offgas, pre- | | |
|---|---|---|---|---|---|---|---|
| | Addition of | Addition of a cocatalyst | | | dominantly | pH of the | |
| Ex- | NaHCO₃ | Type/Amount | TAA con- | Yield *) | O₂ | aqueous | Glass apparatus, |
| ample | (mol) | (mol) | version (%) | (%) | (1) | phase**) | glass blade stirrer |
| 1 | 0.21 | —/— | 90 | 83 | 3 | 8.8 | without steel mesh |
| (Comp.) | | | | | | | |
| 2 | 0.21 | —/— | 14.8 | 12 | 35.9 | 9.9 | with steel mesh |
| (Comp.) | | | | | | | |
| 3 | 0.21 | H₃PO₄/0.06 | 87.3 | 79 | 3.1 | 8.6 | with steel mesh |
| 4 | 0.21 | H₃PO₄/0.06 | 87.5 | 80 | 1.8 | 8.5 | with steel mesh |
| 5 | 0.21 | NaH₂PO₄ *1 H₂O/0.06 | 46.7 | 41 | 17 | 9.5 | with steel mesh |
| 6 | 0.21 | NaH₂PO₄ *1 H₂O/0.15 | 53.3 | 46 | 15 | 9.2 | with steel mesh |
| 7 | 0.21 | Na₂HPO₄ *2 H₂O/0.05 | 47.7 | 42 | 23.1 | 9.6 | with steel mesh |
| 8 | — | NaH₂PO₄ *1 H₂O/0.21 | 62.1 | 49 | 4.3 | 8.0 | with steel mesh |
| 9 | 0.21 | Nitric acid/0.06 | 46.9 | 42 | 21.7 | 9.5 | with steel mesh |
| 10 | 0.21 | H₂SO₄/0.06 | 36.9 | 36 | 24.9 | 9.2 | with steel mesh |
| 11 | 0.21 | HCl/0.06 | 84.2 | 78 | 4.1 | 8.7 | with steel mesh |
| 12 | 0.21 | CH₃COOH/0.06 | 88.7 | 82 | 3.5 | 8.6 | with steel mesh |
| 13 | 0.21 | 2-Ethylhexanoic acid/0.06 | 74.3 | 68 | 2.0 | 9.2 | with steel mesh |

*) Yield (only in the organic phase) of oxidized TAA [II + III] in % of theory based on the TAA used. The total yield (in organic and aqueous phase) in each case corresponded approxiamtely to the TAA conversion.
**)immediately after the end of the addition of hydrogen peroxide at room temperature

### Example 14 (Comparative)

1960 kg of deionized water, 2.5 kmol of NaHCO₃ and 22.5 kmol of TAA were placed in a 20 m³ steel reactor, a temperature of 60° C. was set and 100 kmol of 50% strength aqueous H₂O₂ solution were metered in over a period of 60 hours. The off gas obtained in the process was brought to an oxygen concentration of <7% by addition of N₂ and was passed to appropriate disposal (offgas incineration).

### Example 15

Example 15 was carried out in a manner analogous to Example 14, but the pH of the reaction mixture was maintained at 8-9 during the reaction time of 60 hours by addition of H₃PO₄ (1 kmol).

The results of Examples 14 and 15 are shown in the following table.

| | | | | pH of the | |
|---|---|---|---|---|---|
| | Addition of | Addition of a cocatalyst | TAA conversion | aqueous | |
| Example | NaHCO₃ (kmol) | Type/Amount (kmol) | (%) | phase**) | Reactor |
| 14 | 2.5 | —/— | 36 | 10.5 | steel |
| | | | | | reactor |
| 15 | 2.5 | H₃PO₄/1 | 93 | 8 | steel |
| | | | | | reactor |

**)immediately after the end of the addition of hydrogen peroxide at room temperature

### Example 16 (Comparative)

11.7 mol of deionized water, 0.21 mol of NaHCO₃ and 2.0 mol of triacetonamine-ethylene glycol ketal (TAA-EGK) were placed in a jacketed 11 glass reactor provided with a glass blade stirrer, the reaction mixture was brought to 60° C. by means of a thermostat while stirring (400 rpm) and 2.8 mol of hydrogen peroxide as a 50% strength aqueous solution were added at a uniform rate via a cool dropping funnel over a period of 4 hours. The reactor temperature was maintained at 57-63 C. The offgas formed in the process, which comprised predominantly O₂, was measured volumetrically by the displacement principle.

At the end of the addition, a sample of the two-phase reaction mixture was taken and the organic phase was analyzed by gas chromatography to determine the product composition. The pH of the lower aqueous phase was measured at room temperature.

### Example 17

To simulate H₂O₂-destroying conditions, a piece of steel mesh packing was attached to the glass blade stirrer and the experiment was otherwise carried out as in Example 16.

### Example 18

Example 18 was carried out in a manner analogous to Example 17, but 0.06 mol of H₃PO₄ was used as cocatalyst in addition to the 0.21 mol of NaHCO₃ used.

The results of Examples 16-18 are shown in the following table.

| | | | TAA- | | Offgas, | pH of the | |
|---|---|---|---|---|---|---|---|
| | Addition | | EGK | | pre- | aqueous | Glass |
| | of | Addition of a | con- | | domin- | phase | apparatus, |
| Ex- | NaHCO₃ | cocatalyst | version | Yield*) | antly O₂ | after the | glass blade |
| ample | (mol) | Type/Amount/(mol) | (%) | (%) | (1) | reaction | stirrerr |
| 16 | 0.21 | —/— | 82 | 77 | 6 | 9.2 | without steel |
| | | | | | | | mesh |
| 17 | 0.21 | —/— | 39 | 38 | 22 | 9.7 | with steel |
| | | | | | | | mesh |
| 18 | 0.21 | H₃PO₄/0.06 | 75 | 70 | 5.6 | 9.2 | with steel |
| | | | | | | | mesh |

*)Yield of oxidized TAA-EGK (II + III) in % of theory (only the organic phase) based on the TAA-EGK used. The total yield (in organic and aqueous phase) in each case corresponded approximately to the TAA-EGK conversion

### Example 19 (continuous)

Two glass reactors (A1, A2) which were provided with glass blade stirrers and were connected in series were each charged to the overflow with 0.461 of a reaction mixture corresponding to Example 18, brought to 60° C. and the following feed streams were metered in via metering pumps:

In reactor:

| | A1 | A2 |
|---|---|---|
| Amounts in | (mol/h) | (mol/h) |
| Aqueous NaHCO₃ solution (7.8% strength) | 0.029 | — |
| TAA-EGK: | 0.26 | — |
| Aqueous H₂O₂ solution (50% strength): | 0.40 | 0.10 |
| Aqueous H₃PO₄ (85% strength): | 0.028 | 0.007 |

A mean residence time per reactor of 4 hours can be calculated from the amounts indicated.

The reaction products traveled via the overflow from the reactor A1 into the reactor A2 and from there via the overflow into a glass receiver. The offgas obtained in the process (predominantly O₂) was discharged via a gas collection line and the amount of offgas was determined volumetrically by the displacement principle.

After the equilibrium state had been reached, samples were taken from the two-phase reaction mixtures in the reactors and the glass receiver. The pH of the lower aqueous phase from the glass receiver was firstly measured at room temperature. All samples were diluted with an alcoholic solvent and the now single-phase product mixture was in each case analyzed by gas chromatography to determine the product composition:

| | Reactor: | | |
|---|---|---|---|
| | A1 | A2 | Glass receiver |
| TAA-EGK conversion | 79 | 88 | 89 |
| (%): | | | |
| Yield (%)*⁾: | 78 | 87 | 88 |
| pH of the aqueous phase: | | | 7.7 |
| Amount of off gas (l/h): | | | 1.2 |

*⁾Yield of oxidized TAA-EGK (II + III) in % of theory based on the TAA-EGK used German application 102004023640.2 is incorporated herein by reference in its entirety.

Obviously, numerous modifications and variations of the present invention are possible in light of the above teachings. It is therefore to be understood that within the scope of the appended claims, the invention may be practiced otherwise than as specifically described herein.

1. A process for preparing one or more 4-substituted 2,2,6,6-tetramethylpiperidin-N-oxy compounds of formula (II) or mixture of (II) and one or more 4-substituted 2,2,6,6-tetramethylpiperidin-N-hydroxy compounds of formula (III) 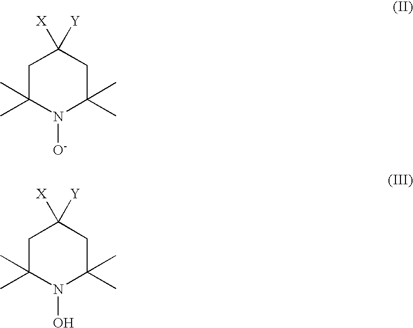
where X and Y may together represent a single O atom or
a cyclic ketal radical of formula 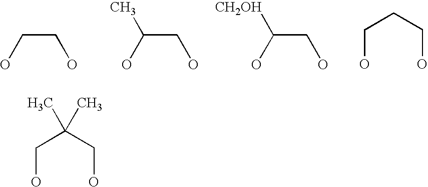
or X and Y may be an open-chain ketal in which X═O—R and Y═O—R′, where R and R′ can be identical or different and are selected from the group consisting of CH₃, CH₂—CH₃, CH₂—CH₂—CH₃, CH(CH₃)—CH₃, CH₂—CH₂—CH₂—CH₃ and CH₂—CH(CH₃)—CH₃, said process comprising:
oxidizing a 4-substituted 2,2,6,6-tetramethylpiperidine of formula (I) 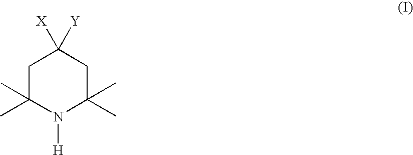
with hydrogen peroxide in the presence of one or more of an alkali metal hydrogencarbonate or an ammonium hydrogencarbonate, and in the presence or absence of a solvent, wherein one or more Brönsted acids having an acid strength greater than that of the hydrogencarbonate is present during the oxidizing.

2. The process as claimed in claim 1, wherein the oxidizing is carried out in the presence of at least one alkali metal hydrogencarbonate selected from the group consisting of lithium, sodium, potassium, rubidium and cesium hydrogencarbonate.

3. The process as claimed in claim 1, wherein at least one of the alkali metal hydrogencarbonate or the ammonium hydrogencarbonate is present during the oxidizing in an amount of from 0.02 to 0.5 molar equivalent, based on the amount of the amine (I).

4. The process as claimed in claim 1, wherein the Brönsted acid is one or more inorganic acids.

5. The process as claimed in claim 1, wherein the Brönsted acid is one or more organic acids.

6. The process as claimed in claim 1, wherein the Brönsted acid is present as a mixture of one or more inorganic acids and one or more organic acids.

7. The process as claimed in claim 4, wherein at least one inorganic acid selected from the group consisting of phosphoric acid, dihydrogenphosphate, hydrogenphosphate, nitric acid, sulfuric acid, hydrogensulfate and a hydrohalic acid, is present during the oxidizing.

8. The process as claimed in claim 5, where the oxidizing is carried out in the presence of one or more Brönsted acids selected from the group consisting of formic acid and a saturated linear or branched, monobasic or polybasic aliphatic carboxylic acid which has from 2 to 12 carbon atoms and may be substituted by O—R or NR²R³, where R¹, R² and R³ can be identical or different and are each hydrogen, an aliphatic or cycloaliphatic, saturated alkyl radical having from 1 to 12 carbon atoms or a carboxyalkyl group (CH₂)ₙCOOH where n=1 to 5 or R² and R³ can together form a saturated, unsubstituted or alkyl-substituted alkylene chain having from 4 to 11 carbon atoms, with the proviso that when R² and R³ are both hydrogen or alkyl or the two together form an alkylene group or when R²=hydrogen and R³=alkyl, the NR²R³-substituted carboxylic acid has to be polybasic.

9. The process as claimed in claim 8, the Brönsted acid is at least one selected from the group consisting of acetic acid, hydroxyacetic acid, methoxyacetic acid, propionic acid, butyric acid, 2-ethylhexanoic acid, oxalic acid, malonic acid, succinic acid, glutaric acid, citric acid, iminodiacetic acid, nitrilotriacetic acid and an acidic amino acid.

10. The process as claimed in claim 5, wherein the oxidizing is carried out in the presence of at least one of a phosphonic acid or a partial salt of a phosphonic acid having the following formula:
Z(PO₃HₙM₂₋ₙ)ₘ
where Z represents one or more, linear or branched alkyl radicals or diradicals which have a total of from 1 to 10 carbon atoms and may contain up to 3 nitrogen atoms; m is from 1 to 6; n is 1 or 2; and M is an alkali metal or NH₄.

11. The process as claimed in claim 1, wherein the reaction is carried out in the presence of 0.01 to 0.5 molar equivalent of the Brönsted acid, based on the amine (I).

12. The process as claimed in claim 1, wherein the process is carried out in the presence of water so that the aqueous phase of a two-phase mixture or the aqueous/organic phase of a homogeneous reaction medium has a pH of from 7.0 to 10.0 after the oxidizing is complete.

13. The process as claimed in claim 12, wherein the reaction is carried out so that the aqueous phase of a two-phase mixture or the aqueous/organic phase of a homogeneous reaction medium has a pH of from 8.0 to 9.6 after the oxidizing is complete.

14. The process as claimed in claim 1, wherein the hydrogen peroxide is an aqueous solution having a concentration in the range from 10 to 90% by weight.

15. The process as claimed in claim 1, wherein the process is carried out in the presence of one or more of an alcohol, diol, ether compound, ketone, aliphatic, cycloaliphatic, aromatic or araliphatic hydrocarbon.

16. The process as claimed in claim 15, wherein the process is carried out in the presence of at least one selected from the group consisting of methanol, ethanol, n-propanol, isopropanol, tert-butanol, isobutanol, n-butanol, methoxyethanol, ethoxyethanol, ethylene glycol, propylene glycol, ethylene diglycol, propylene diglycol, alkyl glycol ethers, 1,3-dioxane, 1,4-dioxane, tetrahydrofuran, acetone, heptane, cyclohexane, ethylcyclohexane, toluene and xylene.

17. The process as claimed in claim 1, wherein the reaction is carried out at from 20° C. to 150° C.

18. The process as claimed in claim 1, wherein the amine (I), the alkali metal hydrogencarbonate, the ammonium hydrogencarbonate, the Brönsted acid and optionally one or more solvents are initially charged into a reactor and the hydrogen peroxide is added stepwise or continuously to the reaction mixture over a period of from 0.1 to 72 hours.

19. The process as claimed in claim 18, wherein the duration of the adding is from 2 to 40 hours.

20. The process as claimed in claim 1, wherein all components are fed simultaneously and continuously into a reactor system.

21. The process as claimed in claim 20, wherein the process is carried out in a reactor system comprising a tube reactor, a reactor cascade having at least 2 reactors, or a combination thereof.

22. The process as claimed in claim 1, wherein the oxidizing is carried out so that the reaction medium comes into contact with one or more metal surfaces.

23. The process as claimed in claim 22, wherein the metal surfaces comprise an alloy of one or more of iron, titanium, zirconium, vanadium, niobium, tantalum, chromium, molybdenum, tungsten, manganese, cobalt, nickel, copper, zinc or aluminum.

24. A process for preparing 4-substituted 2,2,6,6 tetramethylpiperidin-N-oxy compounds of formula (II) or mixtures of (II) and 4-substituted 2,2,6,6-tetramethylpiperidin-N-hydroxy compounds of formula (III) 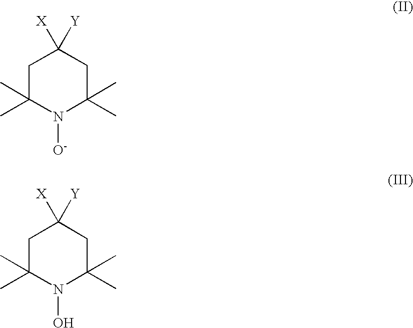
where X and Y together may be a single O atom or a cyclic ketal radical of the following formula 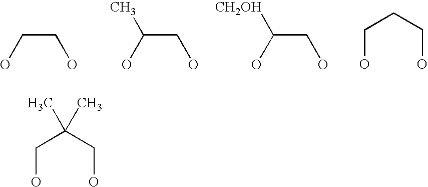
or X and Y may be an open-chain ketal in which X═O—R and Y═O—R′, where R and R′ can be identical or different and can each be CH₃, CH₂—CH₃, CH₂—CH₂—CH₃, CH(CH₃)—CH₃, CH₂—CH₂—CH₂—CH₃ and CH₂—CH(CH₃)—CH₃, said process comprising:
oxidizing a mixture comprising one or more 4-substituted 2,2,6,6-tetramethylpiperidines of formula (I), 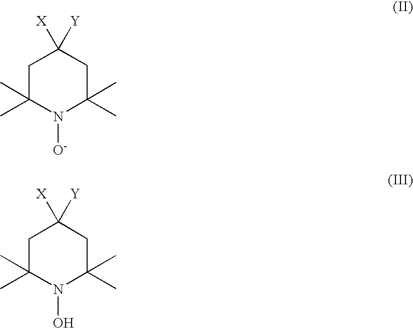
with hydrogen peroxide in the absence of hydrogencarbonate, and
adding at least one of alkali metal dihydrogenphosphate or an ammonium dihydrogenphosphate to the mixture.

25. The process as claimed in claim 6, wherein at least one inorganic acid selected from the group consisting of phosphoric acid, dihydrogenphosphate, hydrogenphosphate, nitric acid, sulfuric acid, hydrogensulfate and a hydrohalic acid is present during the oxidizing.

26. The process as claimed in claim 6, where the oxidizing is carried out in the presence of one or more Brönsted acids selected from the group consisting of formic acid and a saturated linear or branched monobasic or polybasic aliphatic carboxylic acid which has from 2 to 12 carbon atoms and may be substituted by O—R¹ or NR²R³, where R¹, R² and R³ can be identical or different and are each hydrogen, an aliphatic or cycloaliphatic, saturated alkyl radical having from 1 to 12 carbon atoms or a carboxyalkyl group (CH₂)ₙCOOH where n=1 to 5 or R² and R³ can together form a saturated, unsubstituted or alkyl-substituted alkylene chain having from 4 to 11 carbon atoms, with the proviso that when R² and R³ are both hydrogen or alkyl or the two together form an alkylene group or when R²=hydrogen and R³=alkyl, the NR²R³-substituted carboxylic acid has to be polybasic.

27. The process as claimed in claim 6, wherein the oxidizing is carried out in the presence of at least one of a phosphonic acid or a partial salt of a phosphonic acid having the following formula:
Z(PO₃HₙM₂₋ₙ)ₘ
where Z represents one or more, linear or branched alkylic radicals or diradicals which have a total of from 1 to 10 carbon atoms and may contain a total of up to 3 nitrogen atoms; m is from 1 to 6; n is 1 or 2; and M is an alkali metal or NH₄.

28. The process as claimed in claim 1, wherein the oxidizing is carried out in the presence of 0.03 to 0.1 molar equivalent of the Brönsted acid, based on the amine (I).

29. The process as claimed in claim 12, wherein the reaction is carried out so that the aqueous phase of a two-phase mixture or the aqueous/organic phase of a homogenous reaction medium has a pH of from 8.2 to 9.2, after the oxidizing is complete.

30. The process as claimed in claim 1, wherein the hydrogen peroxide is an aqueous solution having a concentration in the range of from 20 to 60% by weight.

31. The process as claimed in claim 1, wherein the hydrogen peroxide is an aqueous solution having a concentration in the range of from 30 to 50% by weight.

32. The process as claimed in claim 1, wherein the oxidizing is carried out at from 50 to 90° C.

33. The process as claimed in claim 1, wherein the oxidizing is carried out at a temperature of from 60 to 80° C.

34. The process as claimed in claim 18, wherein the duration of the adding is from 4 to 10 hours.
